(19) United States
(12) Patent Application Publication
(10) Pub. No.: US 2025/0177647 A1
(43) Pub. Date: Jun. 5, 2025

(54) FLUID DELIVERY DEVICE WITH ACTIVE SAFETY FUNCTION FOR DETECTION AND CONFIRMATION OF ANOMALOUS WIRELESS COMMANDS FROM AUXILIARY DEVICE

(71) Applicant: Becton, Dickinson and Company, Franklin Lakes, NJ (US)

(72) Inventors: Dylan WILSON, Chapel Hill, NC (US); Sean ULRICH, Wake Forest, NC (US); Ronald MOULTON, Woburn, MA (US); Mark WOOD, Sterling, MA (US)

(73) Assignee: Becton, Dickinson and Company, Franklin Lakes, NJ (US)

(21) Appl. No.: 18/834,532

(22) PCT Filed: Jan. 27, 2023
(86) PCT No.: PCT/US2023/011704

§ 371 Date: Jul. 30, 2024

Related U.S. Application Data
(60) Provisional application No. 63/304,710, filed on Jan. 31, 2022.

(51) Int. Cl. A61M 5/172; A61M 5/142; G16H 40/67

(52) U.S. Cl. CPC A61M 5/172; A61M 5/14248; G16H 40/67; A61M 2205/3317; A61M 2205/3576; A61M 2205/502

(57) ABSTRACT

A fluid delivery device (FDD) has a reservoir, a driver assembly, and a controller to controllably deliver fluid from the reservoir into a patient. The controller is configured to support dosing control and other operations of the fluid delivery device by a connected auxiliary device such as a smartphone or other portable remote control device. Active safety features are provided for commands to the fluid delivery device that comprise: (1) at least anomaly detection for inputted FDD commands and prompt for confirmation of anomalous commands prior to commanded operation of the FDD; and (2) an optional secondary communication path or modality for the confirmation that is different from a first communication path or modality used to communicate the command to the FDD.

### CROSS-REFERENCE TO RELATED APPLICATIONS

This application is a 35 U.S.C. 371 National Stage Patent Application of PCT Patent Application No. PCT/US2023/011704, filed Jan. 27, 2023, which claims priority to U.S. Provisional Patent Application Ser. No. 63/304,710, filed Jan. 31, 2022, the entire contents of which are incorporated herein by reference.

### BACKGROUND

### Field

Illustrative embodiments relate generally to active safety features in a fluid delivery device such as a wearable medication infusion patch pump to confirm an anomalous command from an auxiliary device before operation of the fluid delivery device in accordance with the command. Illustrative embodiments relate generally to determining when commands are anomalous using at least one anomaly detection algorithm, requiring a user action to confirm a detected anomalous command before operating the fluid delivery device in accordance with that command, and optionally using a mode of communicating the confirmation to the fluid delivery device that is different from the mode of communicating the command.

### Description of Related Art

Demand for on-body or wearable medical devices and for body area network (BAN) medical devices (e.g., wireless controllers for on-body devices, and smartphones or smart watches with a medical condition management app and/or a health/fitness app) has been increasing, along with an increase in patients' and healthcare providers' desire for better and more convenient patient management of medical conditions such as diabetes. For example, one type of wearable medical device is a wearable medication delivery device that is worn against a patient's skin (e.g., a patch pump with cannula or needle inserted into the patient's skin), or a pump device that can be connected to a patient's belt or placed in a pocket of the user's clothing, for example, and having an infusion set with tubing extending from the pump to an adhesive mount with a subcutaneous cannula.

A wearable medical device can communicate wirelessly with a separate dedicated controller or smartphone (e.g., a smartphone with app configured to wirelessly interface with the wearable medical device for various operations). Bluetooth® Low Energy (BLE), marketed as Bluetooth® Smart, is a wireless technology that provides an effective, low power protocol for wirelessly connecting devices, including devices that run on power sources such as coin cell batteries as can often be the case with wearable medical devices.

A concern with wirelessly controlling a wearable medical device, particularly a device that delivers medication to a patient's body, via another device is security of the wireless control communication link between the medical device and the other device against man-in-the-middle (MITM) and eavesdropping attacks. Of particular concern is security of a wearable medical device against nefarious attacks or unintentional attacks wherein control of the medical device is undesirably altered by another device (e.g., the user's device, or another device that is not the user's intended remote control device, operating in an undesirable manner).

### SUMMARY

A need exists for fluid delivery device and auxiliary device cooperation that safely and securely supports dosing control of the fluid delivery device from the auxiliary device, among other operations. The above and other technical problems are overcome, and additional advantages are realized, by technical solutions provided by the illustrative embodiments of the present disclosure.

In accordance with illustrative embodiments, a fluid delivery device is provided that comprises a reservoir that can contain a fluid to be delivered to a user; a user output port configured to provide a fluid path between the reservoir and the user; a drive assembly configured to controllably drive fluid from the reservoir into the fluid path; a communication interface configured to send and receive signals on a communication path; and a controller connected to the drive assembly and the communication interface and configured to control the drive assembly to deliver a designated amount of fluid from the reservoir to the fluid path. The controller is further configured to analyze a command to operate the drive assembly to determine when the command corresponds to an anomalous command. The controller generates a prompt for confirmation of the command when the command corresponds to an anomalous command, operates the drive assembly in accordance with the command when a reply to the prompt is received and comprises an input corresponding to an affirmative confirmation, and ignores the command when the reply to the prompt is a negative confirmation chosen from an input corresponding to a negative confirmation, and no reply being received within a designated time period after the prompt.

In accordance with aspects of the illustrative embodiments, the controller is further configured to operate the drive assembly in accordance with the command without the prompt for confirmation or the reply with an input corresponding to an affirmative confirmation when the command is not an anomalous command.

In accordance with aspects of the illustrative embodiments, the controller is configured to process a parameter in the command in accordance with at least one anomaly detection algorithm to compare the parameter with a value chosen from a threshold value and a selected range of values, and to determine the command to be an anomalous command if the parameter exceeds the threshold value or is outside the selected range of values and to be normal command if the parameter satisfies a threshold value or selected range of threshold values.

In accordance with aspects of the illustrative embodiments, the parameter level or range is preset, or adapted over time in accordance with operation of the fluid delivery device.

In accordance with aspects of the illustrative embodiments, the controller is configured to process the command by analyzing variables chosen from historical commands to deliver the fluid to the patient and historical disease management data related to the patient to identify a fluid delivery device use pattern corresponding to one or more parameters related to amounts of fluid delivered, when the fluid is delivered, and patient physiological data affected by delivery of the fluid, and to detect when the command corresponds to an anomalous command because a parameter in the command is an outlier relative to one or more of the parameters of the use pattern.

In accordance with aspects of the illustrative embodiments, the controller determines when the command corresponds to an anomalous command by using at least one anomaly detection algorithm.

In accordance with aspects of the illustrative embodiments, the at least one anomaly detection algorithm employs anomaly detection techniques chosen from local outlier factor, histogram-based outlier score, one-class support vector machine, robust covariance, k-nearest neighbor, isolation forests, supervised machine-learning model, unsupervised machine-learning model, and hybrid approach using a semi-supervised machine-learning model.

In accordance with aspects of the illustrative embodiments, the command is communicated to the fluid delivery device from an auxiliary device. The auxiliary device, for example, is chosen from a smartphone, a computer, a laptop, an iPad, and a dedicated, portable remote controller configured to command the fluid delivery device.

In accordance with aspects of the illustrative embodiments, the auxiliary device is connected to the fluid delivery device by a communication modality chosen from WiFi, Bluetooth®, near field communication (NFC), cellular communication, and wired communication, and is configured to remotely control the fluid delivery device by sending commands via the communication modality.

In accordance with aspects of the illustrative embodiments, the auxiliary device is connected to the fluid delivery device by a communication modality, and the command and the prompt are both transmitted using the communication modality. The communication modality is chosen, for example, from WiFi, near field communication (NFC) and Bluetooth®.

In accordance with aspects of the illustrative embodiments, the prompt is transmitted using a second communication modality that is different from a first communication modality used to send the command.

In accordance with aspects of the illustrative embodiments, the first communication modality is chosen from WiFi and Bluetooth®, and the second communication modality is chosen from near field communication (NFC), a magnet and Hall Effect sensor provided to respective ones of the fluid delivery device and an auxiliary device that sends the command to the fluid delivery device, an accelerometer sensor configured to detect a movement of an auxiliary device relative to the fluid delivery device, and a switch on the fluid delivery device.

In accordance with aspects of the illustrative embodiments, the controller comprises a communication processor device and a safety processor device. The communication processor device establishes secure messaging with an auxiliary device connected to the fluid delivery before passing a message signal comprising the command from the auxiliary device to the fluid delivery. The safety processor device performs anomaly detection of the command.

In accordance with aspects of the illustrative embodiments, the communication processor device and the safety processor device are both provided within the fluid delivery device.

In accordance with aspects of the illustrative embodiments, the fluid delivery device further comprises a processing device separate from and coupled to the controller. The processing device is configured to analyze the command to operate the drive assembly to determine when the command corresponds to an anomalous command, and to provide an indication to the controller when the command is determined to correspond to an anomalous command. The controller is configured to generate the prompt for confirmation of the command when the command corresponds to an anomalous command, operate the drive assembly in accordance with the command when a reply to the prompt is received and comprises an input corresponding to an affirmative confirmation, and ignore the command when the reply to the prompt is a negative confirmation chosen from an input corresponding to a negative confirmation, and no reply being received within a designated time period after the prompt.

Additional and/or other aspects and advantages of illustrative embodiments will be set forth in the description that follows, or will be apparent from the description, or may be learned by practice of the illustrative embodiments. The illustrative embodiments may comprise apparatuses and methods for operating same having one or more of the above aspects, and/or one or more of the features and combinations thereof. The illustrative embodiments may comprise one or more of the features and/or combinations of the above aspects as recited, for example, in the attached claims.

### BRIEF DESCRIPTION OF THE DRAWINGS

The above and/or other aspects and advantages of embodiments of the illustrative embodiments will be more readily appreciated from the following detailed description, taken in conjunction with the accompanying drawings, of which:

FIG. 1 is a perspective view of a wearable fluid delivery device (FDD) in communication with an auxiliary device (e.g., smartphone with FDD control software app or dedicated FDD controller) in accordance with an example embodiment;

FIG. 2 is a perspective view of the FDD of FIG. 1 with the cover removed;

FIG. 3 is a block diagram of example components of a FDD in accordance with an example embodiment;

FIG. 4 is a block diagram of firmware provided in a controller in a FDD in accordance with an example embodiment;

FIG. 5 is a block diagram of example components of an auxiliary device in accordance with an example embodiment; and

FIG. 6 is a flow chart illustrating operations in an example method of providing active safety features functions for a FDD that detect and confirm an anomalous command from an auxiliary device before operation of the FDD in accordance with the command in accordance with an illustrative embodiment.

Throughout the drawing figures, like reference numbers will be understood to refer to like elements, features and structures.

### DETAILED DESCRIPTION OF ILLUSTRATIVE EMBODIMENTS

As will be appreciated by one skilled in the art, there are numerous ways of carrying out the examples, improvements, and arrangements of an active safety function provided to a fluid delivery device (FDD) that receives wireless commands from an auxiliary device in accordance with embodiments disclosed herein. Although reference will be made to the illustrative embodiments depicted in the drawings and the following descriptions, the embodiments disclosed herein are not meant to be exhaustive of the various alternative designs and embodiments that are encompassed by the disclosed technical solutions, and those skilled in the art will readily appreciate that various modifications may be made, and various combinations can be made without departing from the scope of the disclosed technical solutions. For example, embodiments for the present disclosure are described with respect to remote control of infusion of insulin or another medicinal fluid for purposes of regulating the user's blood glucose levels. However, numerous other types of medicines can be used by the FDD described in the example embodiments such as, but not limited to, pain relief drugs, hormone therapy, blood pressure treatments, anti-emetics, osteoporosis treatments, or other injectable medicines into the tissue or vasculature of a human or animal patient.

Insulin dosing is a critical task in the daily life of a Type 1 or Type 2 diabetic. Some existing insulin infusion pumps are connected to an auxiliary device (e.g., a dedicated proprietary controller) via a wireless or wired communication interface. These pumps allow users to control dosing from the connected auxiliary device. In the future, as regulation of medical devices permits, dosing may be allowed from a user's personal smartphone via wireless communication (e.g., Bluetooth® protocol). FDDs can be manually operated to set and commence dosing, or can be set to automatically deliver a designated amount of medication at a designated time. Either way, a user interface for entering desired dose amount and/or time of delivery can be on an auxiliary device such as a personal smartphone with control functions for the FDD, or on a dedicated wirelessly connected remote controller.

While Bluetooth® can provide a secure communication path or channel between a FDD and an auxiliary device, significant harm to the user could occur in the case of unintended doses (e.g., particularly with insulin dosing), whether through user error operating the FDD or auxiliary device, or by a malicious actor. For example, FDDs need to be designed to prevent someone from purposefully interfering with or altering the communication channel between a FDD and an auxiliary device in a manner that causes the wrong command to be communicated to the FDD, or prevents a needed command from being communicated to the FDD, such that potentially harmful operation of the FDD could occur (e.g., dose size or timing that is outside an acceptable designated range or otherwise fails to satisfy a designated parameter level or range).

A technical problem therefore exists for cooperation between a FDD and an auxiliary device to safely and securely support dosing control of the FDD from the auxiliary device. This technical problem and other problems are overcome and advantages are realized by illustrative embodiments described herein. Different example embodiments of the present disclosure comprise: (1) at least an active safety feature of anomaly detection for inputted FDD commands and prompt for confirmation of anomalous commands prior to commanded operation of the FDD; and (2) an optional secondary communication path or modality for the confirmation that is different from a first communication path or modality used to communicate the command to the FDD. In accordance with another aspect of example embodiments, operations of the FDD controller can be implemented using communication channel interface firmware and safety firmware, wherein the communication channel interface firmware performs authentication to securely receive messages from an auxiliary device and forwards commands received via authenticated messages to the safety firmware, and wherein the safety firmware performs processing of the commands to ensure fluid delivery amounts and/or times of fluid delivery satisfy designated safety parameters. The communication channel interface firmware and the safety firmware can be provided in the same processing unit or in separate processing units.

As described below, one or more anomaly detection algorithm(s) can be employed at the FDD 10, or at the auxiliary device 63, or at another remote device connected to the FDD 10 and/or the auxiliary device 63, to process an inputted command (e.g., a dosing command) to determine if it is a normal command or an anomalous command before the command is acted upon by the FDD. For example, a normal command can be a command to operate the FDD according to an inputted parameter value that satisfies a designated parameter level or range for that particular patient and/or that particular FDD 10. By contrast, an anomalous command can be, for example, a command to operate the FDD according to an inputted parameter value that fails to satisfy a designated parameter level or range for that particular patient and/or that particular FDD 10. Example embodiments of an active safety feature of anomaly detection with respect to an operation command for a fluid delivery device are described below. For example, one or more anomaly detection algorithms can be used such as statistical methods or Hidden Markov models, among others, to determine if a commanded dose amount is anomalous or not. If the computing power or processing speed and the memory available on the FDD are limited (e.g., by the processing speed, power usage and/or cost of the controller, data storage and/or power components provided in a disposable FDD), the implementation of anomaly detection can be optimized to that environment. In an optimized embodiment using a disposable or partially disposable FDD 10, a user's disease management model and/or associated parameters are transferred to each new FDD 10 as the old FDD 10 is replaced and disposed of so that this user data does not have to be relearned by the controller in the new FDD 10.

With regard to the optional secondary communication path or modality for confirmation that is different from the communication path or modality used to input the command, different embodiments can include, but are not limited to, features on the pump such as a near field communication (NFC) interface, an accelerometer or other sensor used to detect a move and/or tap pattern of an auxiliary device relative to the FDD, a voice command (e.g., a microphone and phone app that can perform speech to command conversion), Bluetooth® signal strength (e.g., bringing smartphone or other auxiliary revise very close to the FDD), a fingerprint sensor, a physical user interface button, a magnet and Hall Effect sensor switch (e.g., a magnet or corresponding Hall Effect sensor provided in a respective one of custom jewelry such as a ring, bracelet, or watch that can be brought in close proximity to the other one of the magnet and corresponding Hall Effect sensor provided at a FDD 10 to indicate a user's confirmation of a command to the FDD), among other confirmation communication paths or modalities.

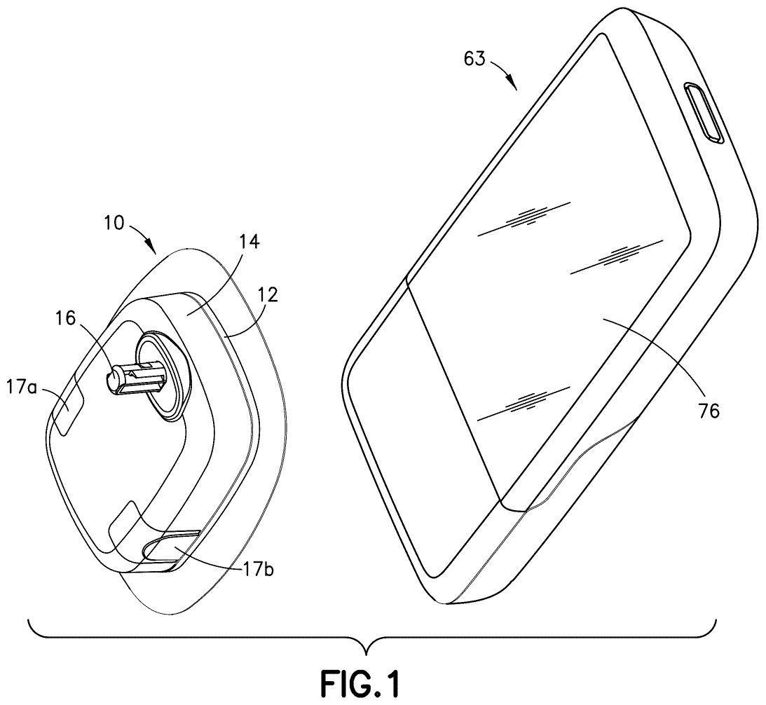

FIG. 1 is a perspective view of an example wearable FDD 10 and an auxiliary device 63 in accordance with an example embodiment. The FDD 10 comprises a baseplate 12, a cover 14, and an insertion mechanism 16 in an undeployed position. Also provided is a user input interface 17 (e.g., button 17a and/or button 17b) that can used to provide a user input (e.g., by depressing the button 17a and/or button 17b) to indicate a user's desire to commence an operation or function at the wearable fluid delivery device 10. The user requested operation or function can depend on the current operational mode or state of the device 10. For example, the user interface 17 (e.g., button 17a and/or button 17b) can be activated when the device is in a manufacturing or shelf mode (e.g., pre-user initialization) to wake-up the device from a power save mode and to commence initialization. As a further example, the user interface 17 (e.g., button 17a and/or button 17b) can be activated when the device is in a delivery mode to indicate that delivery of a bolus is desired. Prior to wake-up and initialization, a reservoir in the FDD 10 can be filled with a fluid (e.g., a drug such as insulin) by a user inserting a needle of a filled syringe into a fill port (not shown) provided in the baseplate 12 that has an inlet fluid path from the fill port to the reservoir. It is to be understood that the FDD 10 can be filled with a fluid (e.g., drug) using different mechanisms and methods.

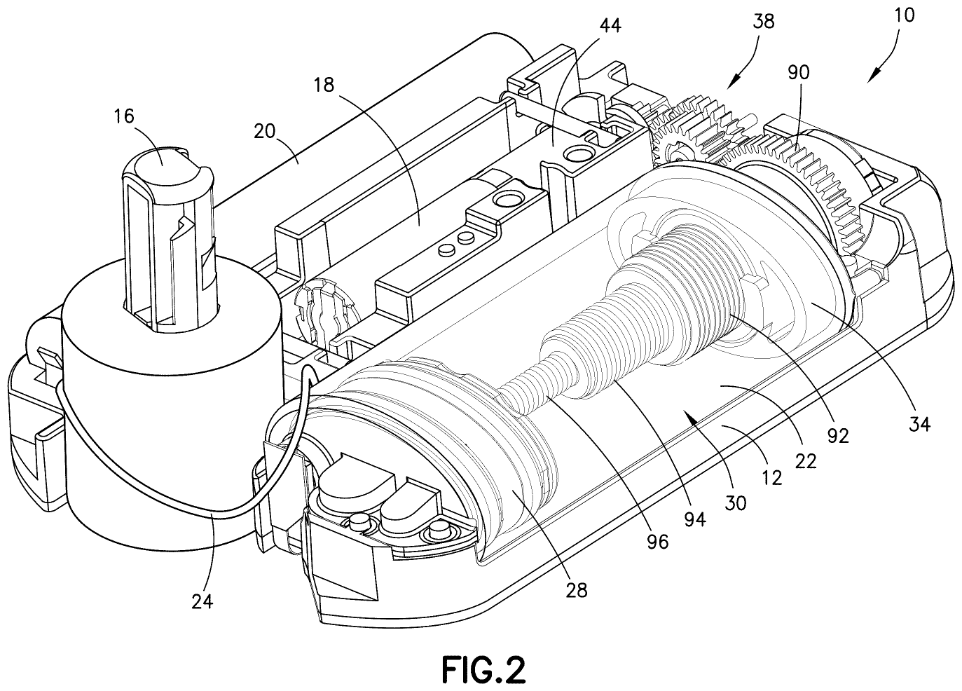

FIG. 2 is a perspective view of the FDD 10 of FIG. 1 with the cover 14 removed. The baseplate 12 supports the insertion mechanism 16, a motor 18, a power source such as a battery 20, a control board (not shown), a motor housing and gearbox 44, and the reservoir 22 or container for storing a fluid to be delivered to a user via an outlet fluid path 24 from an outlet port of reservoir to the insertion mechanism 16. The reservoir 22 can also have an inlet port connected via an inlet fluid path to a fill port (e.g., provided in the baseplate 12). In the illustrated example of a FDD 10, the reservoir 22 contains a plunger 28. The proximal end of the reservoir 22 is also provided with a plunger driver assembly 30 connected to the plunger and having plural telescopic nested screws 92, 94, and 96, a gear anchor 34, an outermost drive screw 90 that is rotated via a gear train 38 connected to the motor 18. Although a gear train 38 is shown for illustrative purposes, the drive mechanism can be gears, ratchets, or other methods of inducing rotation from a motor.

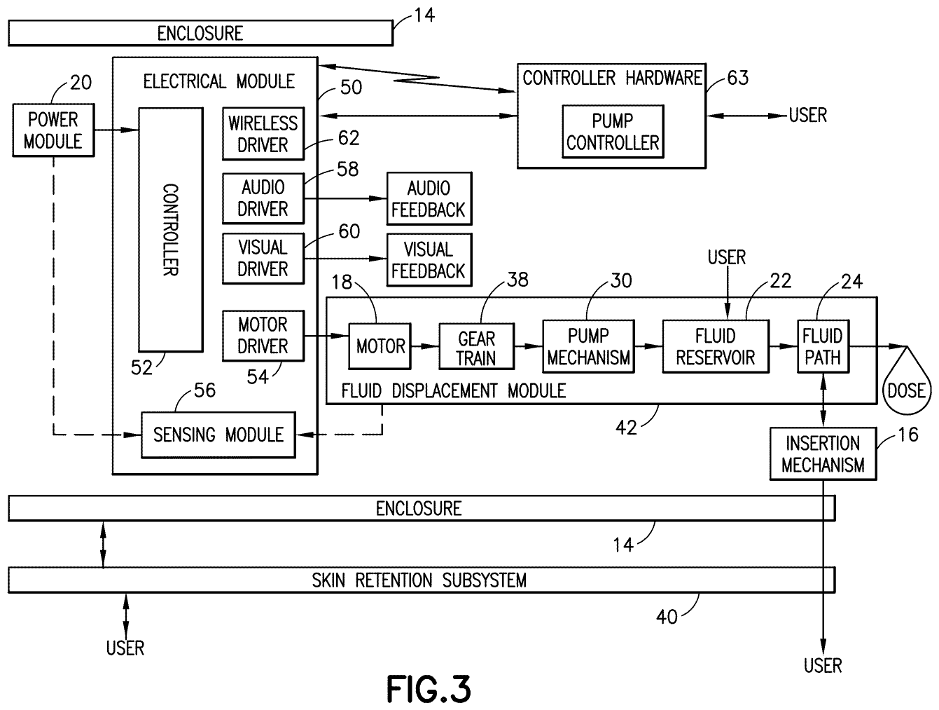

FIG. 3 is a block diagram of example components of a FDD 10 in accordance with an example embodiment. The housing or enclosure of the FDD 10 is indicated at 14. The FDD 10 has skin retention subsystem 40 such as an adhesive pad to connect the FDD 10 to a user's skin. The FDD 10 further comprises the reservoir 22, the insertion mechanism 16, and a fluid displacement module 42 that can include the motor 18, gear train 38, pump mechanism (e.g., plunger driver assembly 30), and outlet path 24. The example FDD 10 further comprises electrical components such as a power module (e.g., battery 20), a user input 17 (e.g., button 17a and/or 17b), and an electrical module 50 comprising a controller 52, a motor driver 54, optional sensing module 56 to sense fluid flow conditions (e.g. occlusion or pump mechanism runaway), optional audio driver 58 (e.g., to indicate dosing in progress, low reservoir, occlusion, successful pairing with external device, or other condition via an audible alarm such as a buzzer), and an optional visual driver 60 to provide visual feedback via a light emitting diode(s) and/or optional tactile driver to provide tactile feedback via a vibration component, and a wireless driver 62 for wireless communication between the FDD and a remote pump control device 63 (e.g., an auxiliary device such as a smartphone or a dedicated controller). With regard to the sensing module 56, the fluid delivery device can be provided, for example, with one or more encoders to provide feedback of the drive mechanism (e.g., plunger driver assembly 30) for indexing and pump mechanism runaway prevention purposes.

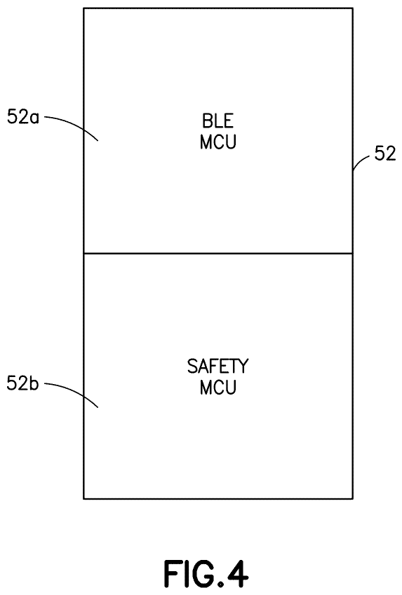

FIG. 4 is a block diagram of an example controller 52 in a FDD 10 comprising a BLE microcontroller unit (MCU) 52a and a Safety MCU 52b in accordance with an example embodiment. The Safety MCU 52b can be an independent microcontroller that monitors motor current, one or more encoders, or other drive assembly sensing device indicated at 56 in FIG. 3 while communicating to the BLE MCU 52a to ensure proper drive assembly operation for desired fluid displacement or pump operation. The Safety MCU 52b and BLE MCU 52a are each provided with firmware. The Safety MCU 52b and BLE MCU 52a can each have the ability to shut down the motor 18 in the event of a fault condition. In accordance with an advantage of this example embodiment, the Safety MCU 52b can be configured to shut down the pump motor 18 if the actions of the BLE MCU 52b violate designated safety parameters to provide improved safe and secure dosing control of the FDD 10 from an auxiliary device 63, among other functions.

One of the main jobs of the BLE MCU 52a firmware is to keep track of the FDD 10's operational modes. The function of the BLE MCU 52a firmware can therefore depend on what mode the FDD 10 is in. Example FDD 10 operational modes tracked by the BLE MCU 52a firmware are: manufacturing, fill, attachment, active, fault, deactivated. The active mode is for fluid delivery (e.g., delivery of a drug from the reservoir 22 to a user). During the active mode, an auxiliary device 63 can transmit the user's basal schedule and bolus doses. The BLE MCU 52a firmware monitors the current time and uses that to determine when a basal delivery is needed. Commands from the auxiliary device 63, or from a FDD user button 17a and/or 17b are used to deliver a bolus. The firmware monitors for device faults such as: uncommanded movements (e.g., drive assembly 30 runaway), occlusions, drive train 38 failures, and low battery conditions. Feedback is provided to the user based on an LED and/or buzzer as shown connected to their respective drivers indicated at 60 and 58 in FIG. 3, and/or through the auxiliary device 63. Unrecoverable faults cause a transfer to the fault mode. Once the reservoir 22 is empty and acknowledged by the user, a transfer can be made to a deactivated mode, for example.

The FDD 10's Safety MCU 52b firmware can have the following modes: pre-delivery mode, delivery mode and fault mode. The firmware can allow the motor 18 to be run without much restriction for manufacturing testing, fill level seeking, and priming to occur. Once pre-delivery mode is completed, the Safety MCU 52b firmware enters into a delivery mode. While in the delivery mode, the Safety MCU 52b monitors for too high of a basal rate and for boluses that are too large or too frequent, and removes power from the motor 18 drive assembly 30 should any of these failure conditions be detected. For example, the ability of the Safety MCU 52b firmware to assure safety is based on some limits for the amount of insulin which can be delivered over various periods of time. In accordance with an example embodiment, the BLE MCU 52a digitally communicates to the Safety MCU 52b. For example, the BLE MCU 52a passes messages with commands to operate the drive assembly 30 (e.g., a command which indicates the patient's intended dose rate) from the auxiliary device 63 to the Safety MCU 52b, along with auxiliary device-generated message authentication. For example, the BLE MCU 52a firmware can be configured to perform security operations such as certification/encryption to securely pair the FDD 10 to an intended auxiliary device 63 (e.g., using custom certification, public/private key exchange, or mutual authorization with unique identifiers (IDs) assigned to and exchanged between a FDD 10 and intended auxiliary device 63). The Safety MCU 52b firmware can be configured to use more controlled or stricter limits on the amount dosed than the amounts provided in a command transmitted from the auxiliary device 63 by (1) comparing dose parameters provided with the inputted command to thresholds related to designated dosing amounts and/or times of delivery; and (2) deferring operation of the FDD 10 in accordance with the inputted command until and if the inputted dose parameters are determined to satisfy safety thresholds for normal operation, or a confirmation is received in response to a prompt requesting confirmation of an anomalous operation from inputted dose parameters that fail to satisfy designated thresholds. This two-factor process for processing inputted commands received from the auxiliary device 63 prior to operating the FDD 100 in accordance with the commands improves the safety of operating the FDD 10 using an intended connected remote control device 63, and protects against malicious actor attacks on the communication path between the FDD 10 and a remote control device that may not be discernible by the intended user of the FDD.

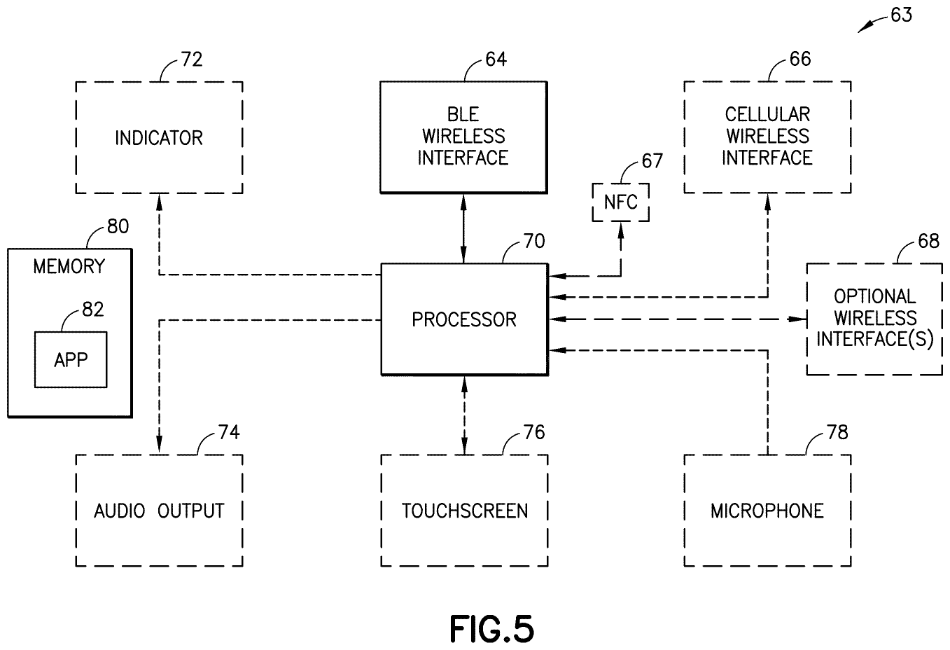

FIG. 5 is a block diagram depicting an example auxiliary device 63. The auxiliary device 63 is described as a smartphone with a FDD control app 82, but it is understood that the auxiliary device 63 can be a dedicated medical management device (e.g., a proprietary wireless remote controller configured for operation with the FDD 10) or other portable, handheld device (e.g., iPad, laptop) or wearable device (e.g., a connected watch, ring or bracelet with software app operable to command the FDD 10). The auxiliary device 63 comprises a processor 70, and a memory 80 that can store a FDD 10 remote control app 82 in accordance with an illustrative embodiment, along with other device data, images and apps. The auxiliary device 63 can have one or more wireless communication interface(s) such as a Bluetooth®-enabled wireless communications interface 64, an optional cellular communications interface 66 and/or other optional wireless communication interface(s) 68 such as a near field communication (NFC) interface, for example, to wirelessly communicate (e.g., via NFC, Bluetooth® connectivity, or another short-range wireless connection, or via radio frequency (RF) or Wi-Fi connectivity, or other wireless connection, or a wired connection, with the FDD 10 to facilitate remote control of the FDD pump driver assembly 30 by a user operating the auxiliary device 63. The auxiliary device 63 can also have different user interfaces such as one or more of a microphone 78, indicator 72 (e.g., light emitting diode(s), or a haptic or vibration indicator device), a touchscreen 76 or other display device that generates graphical user interface (GUI) screens, optional keypad or other user input device (not shown), and an audio signal output device 74 (e.g., a speaker or buzzer).

In addition, the auxiliary device 63 or other device connected to the auxiliary device 63 can have a dosage calculator application that can calculate the recent rate of change in the user's blood glucose level and can use this rate of change information as a parameter in the calculation of a suggested bolus dosage of insulin (or another medication) for the user. In some embodiments, the dosage calculator application can be configured to determine basal rate dosages of insulin (or another medication) along with user-initiated bolus dosages. The auxiliary device 63, or other device connected to the auxiliary device 63, can be configured for a user to enter a carbohydrate value indicative of a meal for use by the dosage calculator application. The suggested bolus dosage or basal rate dosage can then be provided in a command sent from the auxiliary device 63 to the FDD 10, and subjected to anomaly detection and confirmation is anomalous, in accordance with aspects of illustrative embodiments of the present disclosure.

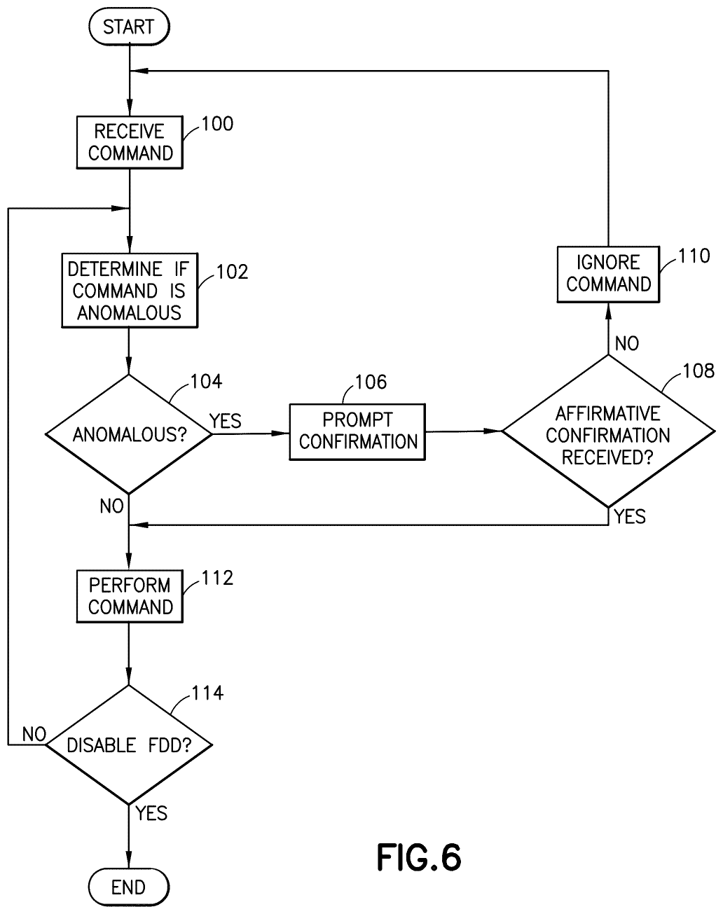

With reference to FIG. 6, an example method of providing active safety functions for a FDD 10 is described that detects and confirms an anomalous command from an auxiliary device 63 before operation of the FDD 10 in accordance with the command, in accordance with an illustrative embodiment. As described above in connection with FIGS. 3 and 5, the control circuitry (e.g., controller 52) of a FDD 10 (e.g., an infusion pump) that includes a wireless or wired communication interface (e.g., wireless driver 62) can implement a process 100 of receiving commands from an auxiliary device 63, and controlling the pump mechanism 30 in accordance with received commands. This process 100, however, is not limited to any particular fluid delivery device.

In accordance with the process 102 in FIG. 6, a command inputted at the auxiliary device 63 and received from the auxiliary device 63 is processed to determine if it is anomalous in accordance with an advantageous aspect of the example embodiment. For example, the controller 52 can be configured to determine whether the received command entered into the auxiliary device 63 is normal (e.g., parameters in the command satisfy a designated parameter level or range or otherwise within acceptable parameters), such as whether a bolus request for an infusion pump is within an acceptable bolus range, as indicated at 104. If the command is normal, then the controller 52 executes the command such as by operating the pump mechanism 30 in accordance with the command, as indicated by the process 112 in FIG. 6. On the other hand, if the controller 52 determines that the received command entered into the auxiliary device 63 is anomalous (e.g., parameters in the command fail to satisfy a designated parameter level or range or are otherwise not within acceptable operation parameters for the FDD 10 or within the user's disease management protocol for using the FDD 10), then the controller 52 generates or otherwise controls generation of a prompt for the user to indicate confirmation of the inputted command, as indicated by the process 106.

With continued reference to FIG. 6, if a user confirmation of the anomalous command is received, then the command is performed as indicated at 112; otherwise, the command is ignored by the controller 52 and not performed at the FDD 10 as indicated at 110. For example, after receiving confirmation from the user to implement a change associated with an inputted command from the auxiliary device 63, the controller 52 can control components of the FDD 10 to change operation in accordance with the user's confirmed command. As a further example, when the user confirms a change to the pump mechanism 30 related to a requested bolus dosage in an inputted command, the controller 52 controls the pump mechanism 30 to deliver the corresponding bolus dispensation of insulin from the reservoir 22. Received commands are processed to detect anomalies until that FDD 10 is disabled (e.g., reaches end of life because the reservoir 22 is empty, or malfunctions, or other shut-down condition) as indicated at 114.

With regard to the receive command process 100 in FIG. 6, a processor (e.g., a controller 52, and optionally a BLE MCU 52a) can be configured to determine if the auxiliary device 63 is authenticated or otherwise authorized to send commands to the FDD 10. A memory accessible to the processor stores information needed for a FDD 10 to securely communicate with an approved or intended remote control device(s) 63, as well as information pertaining to acceptable parameters for use of the FDD 10 on a patient. As stated above, the processor can implement operations such as certification/encryption to securely pair the FDD 10 to an intended auxiliary device 63 (e.g., using custom certification, public/private key exchange, or mutual authorization with unique identifiers (IDs) assigned to and exchanged between a FDD 10 and intended auxiliary device 63).

With further reference to anomaly detection process 102 in FIG. 6, determining if a command for a FDD 10 is normal or anomalous can be performed by a processor such as the controller 52 at the FDD 10 (e.g., optionally a Safety MCU 52b), or by a different device. In accordance with an advantageous embodiment, the process 102 is performed at the FDD 10, and employs disease management parameters (e.g., dose limits, delivery time settings, user physiological data, among other settings) that can be patient-specific and/or device-specific, and can be predetermined or dynamic (i.e., adapted over time such as via machine learning). The disease management parameters can be stored at the FDD 10 or at a separate storage device. If stored at the FDD 10, the stored disease management parameters can be transferred to a new FDD 10 when a disabled FDD is replaced. A processor (e.g., controller 52), at step 102, can review a specific operation request in a received command in view of acceptable operation parameters or guidelines stored in a memory to determine if the operation is permissible. For example, if an operation request in a received command is for delivery of a bolus of insulin or other medical fluid, the processor can determine, for example, whether the size of the bolus is permissible, e.g., is below a maximum bolus value, and is being requested at an acceptable time after a previous bolus request. Similarly, a request within a command to begin or modify basal insulin or other medical fluid delivery can be reviewed to determine if it is within an acceptable range.

In accordance with beneficial aspects of the example embodiments, commands to the FDD 10 are processed according to the process at step 102 (i.e., to determine if the command is for an anomalous change of operation in the FDD 10) regardless of how the command is received. Thus, commands manually inputted by the user or automatically set-up by the user, or received by a malicious attacker, are all subjected to the active safety feature of anomaly detection prior to FDD 10 operation in accordance with the command. The example embodiments are therefore advantageous over a medication delivery device that does not perform anomaly detection, and may only require user confirmation with each user inputted command, and therefore not detect a malicious attacker's wireless interference or nefarious command of which the user is unaware.

Regarding the prompt generation process 106 in FIG. 6, a processor or control circuitry such as controller 52 generates or causes generation of a prompt for the user to confirm an anomalous command, that is, to confirm the user's intent to change the operation of the FDD 10 in accordance with an anomalous command before the processor actually implements the change. This prompt is advantageous over any medication delivery device that requires a user to confirm every inputted command and therefore is less convenient and more cumbersome for the user.

As an example of a prompt per process 106, the processor (e.g., controller 52) can cause generation of a textual prompt on a display 76 at the auxiliary device 63 via a response message transmitted to the auxiliary device 63 via the same communication modality used to receive the command (e.g., a BLE channel) or via a different communication modality. The text prompt can provide a description of the potential change in operation requested via the inputted command (e.g., “Bolus Initiated from Device X; Deliver Requested Bolus of 4.00 Units?”). Alternatively or additionally, other techniques can be used for prompting the user to confirm the user's intent to change the operation of the FDD 10 via an anomalous command. For example, the FDD 10 can provide a visual prompt (e.g., a flashing light), an auditory prompt (e.g., a buzzer) and/or a tactile prompt (e.g., vibration). Alternatively or additionally, any single or combination of these visual, auditory and tactile prompts can be generated at the auxiliary device 63. As a further example, a user can send confirmation of an anomalous command by pressing a button 17a and/or 17b on the FDD 10, or a button on a user input interface on the auxiliary device 63 (e.g., a pushbutton or other input switch, or a touchscreen 76). As described below, the user can optionally provide confirmation of an anomalous command by bumping the auxiliary device 63 against the FDD 10.

In accordance with an aspect of example embodiments in the present disclosure, a FDD 10 receives a confirmation via an optional secondary communication path or modality that is outside of a primary communication path or modality (e.g., a Bluetooth® channel) between the FDD 10 and an auxiliary device 63 (e.g., a remote controller) to confirm a commanded dose amount before it is given. Various example embodiments can be implemented to accommodate example user preferences. In one example embodiment, physical (or very close) access of the auxiliary device 63 to the pump 10 is necessitated in order for the pump 10 to receive a user's confirmation of an anomalous command from the auxiliary device 63. This proximity requirement for an auxiliary device 63 to access the confirmation communication path with the FDD 10 limits the ability for malicious actors to remotely administer unintended doses via an auxiliary device or other device, and limits the likelihood that the user mistakenly doses herself. Other example embodiments for implementing an optional second confirmation communication path or modality can be, but are not limited to, an accelerometer used with a “move/tap” pattern, a voice command (e.g., a microphone and phone app that can perform speech to command conversion), Bluetooth signal strength (e.g., bringing a smartphone 63 very close to a FDD 10), a fingerprint sensor on the smartphone 63, or a magnetic switch/Hall Effect sensor combination (e.g., a magnet or Hall Effect sensor provided in a jewelry piece such as a ring, bracelet, or watch that can be brought close to, respectively, the other one of a Hall Effect sensor and magnet provided in the FDD 10).

In accordance with one example embodiment, a FDD 10 is implemented as an insulin pump with a Near Field Communication (NFC) interface 65 (FIG. 3) that can be controlled by an auxiliary device 63 such as a smartphone with NFC capability and running a pump control app 82. The pump 10 receives commands from the smartphone 63 over a Bluetooth™ low energy (BLE) link, for example. As described above in connection with FIG. 6, the pump 10 runs one or more anomaly detection algorithms and learns the user's normal disease management pattern(s) (e.g., fluid delivery amounts, rates or times of delivery and physiological and other factors that influence dose amounts) over time. When a wireless command is sent to the pump 10 from the smartphone 63, the anomaly detection algorithm(s) determines if the command is considered anomalous. If the command is deemed by the anomaly detection algorithm(s) to be anomalous, the pump control app 82 is notified to generate a user prompt via the smartphone 63. In response to the prompt, the user provides confirmation of an anomalous command using a secondary confirmation method such as a NFC swipe of the smartphone 63 or its connected watch or other connected jewelry to the pump NFC interface 65, or physical button press 17a and/or 17b on the pump to confirm the command. If the user provides the confirmation, the command is executed at the FDD 10. If no confirmation is provided, the command is ignored.

In some embodiments, the FDD 10 can detect a bump with the auxiliary device 63 via its NFC circuit 65 and/or an accelerometer (not shown). As one example, the bump from a smartphone 63 against the pump 10 (e.g., directly or indirectly such that both devices 10 and 63 undergo a detectable bump impact) can cause a simultaneous data exchange between the NFC circuit 65 integrated in the pump 10 and an NFC circuit 67 in the smartphone 63. The data exchange can provide a confirmation signal to the pump 10 indicating that the user has confirmed acceptance of a suggested bolus dosage deemed by the process 102 to be anomalous. In some embodiments, accelerometers in the pump 10 and the smartphone 63 can operate in conjunction with the NFC circuits 65 and 67 to supplement criteria used for activating communications between the NFC circuits 65 and 67. In other words, while in some embodiments NFC communications are initiated based merely on proximity between the NFC circuits 65 and 67, in other embodiments a threshold movement of the pump 10 and/or the smartphone 63 needs to be detected in order to activate NFC communication and therefore confirmation of an anomalous command via this secondary confirmation communication modality.

Regardless of whether a FDD 10 and an auxiliary device 63 employ a secondary confirmation communication modality, the example embodiments described herein are advantageous over other fluid delivery systems that employ an intermediary device between a medical fluid delivery device and a remote controller therefor as a safety measure. The example embodiments can use a primary channel to send commands to a FDD 10 from an auxiliary device 63 acting as a remote controller and therefore without an additional intermediary device that adds complexity to the fluid delivery system and is more cumbersome to the user. Also, example embodiments that employ a secondary confirmation communication modality that is different from the primary command communication modality are more secure than a system wherein an intermediary device receives command and confirmation communications from both a remote device and a medication delivery device using the same communication modality.

As stated above and with further reference to the process 102 in FIG. 6, the anomaly detection algorithm(s) can be executed from an FDD 10 such as a drug delivery infusion pump, or on an auxiliary device 63 such as a smartphone, or on another device accessible via the FDD 10 and/or the auxiliary device 63. As described above in connection with FIG. 6, one or more processors in the FDD 10 or other device runs one or more anomaly detection algorithm(s) and learns the user's normal disease management pattern(s) (e.g., fluid delivery amounts, rates or times of delivery and physiological and other factors that influence dose amounts) over time. In accordance with example embodiments, thresholds are set to determine if command anomalies exist based on, for example, the user's pattern of commands and optionally other data such as default settings for amounts of drug (e.g., insulin) to be delivered and times for delivery of the drug, to achieve a very low false positive rate and thereby avoid annoying the user and adding burden to the disease management process. Furthermore, in accordance with an aspect of example embodiments in the present disclosure, the detection of an anomalous command can be based on one or more conditions chosen from preset thresholds, user preferences, historical and/or predicted user disease management data, and results generated via one or more anomaly detection algorithms. As a further example, confirmation of a suspected anomalous command can be configured to only be prompted per process 106 when a commanded dose is outside of the 95% confidence interval for that user at that time of day.

There are several anomaly detection techniques, such as local outlier factor, histogram-based outlier score, one-class support vector machine, robust covariance, k-nearest neighbor, and isolation forests. These algorithms can find anomalies using two methods such as outlier detection and novelty detection. Outlier detection, also known as unsupervised anomaly detection, uses a training data set of generally unlabeled data and attempts to find values that are far from the other seemingly normal values. Novelty detection, also known as semi-supervised anomaly detection, is used to detect new values that might be anomalies. As another example, a hybrid approach can be used that combine anomaly detection and model-based method(s) by including, among the considered feature set, the prediction residuals obtained using personalized predictive models identified using a single patient's data.

Unlike supervised machine-learning, unsupervised machine-learning does not require labeled data (e.g., a training set of data deemed normal and anomalistic to learning procedure to classify data as an anomaly or normal), but are based instead only on the observation of past examples of data (e.g., historical data), and new data are detected as anomalous if they differ significantly from data previously observed. In general, a combination of the above-referenced information about a user's historical and/or predicted user disease management data can be provided as input to machine learning models that use supervised or unsupervised training methods to configure an artificial intelligence system to output categorical or quantitative output for detecting events such as anomalies in commands for an FDD 10.

The following is a non-limiting example of an anomaly detection algorithm employing an unsupervised machine-learning model to determine if a command is anomalous per process 102 in FIG. 6 in accordance with an example embodiment. As an initial step, blood glucose measurements, meal announcements, and insulin injection information are collected. At respective points in time as the patient uses the FDD 10, feature extraction occurs whereby incoming data are processed (e.g., measured blood glucose values, insulin amounts delivered, optional carbohydrates entered) and numerical features describing patient status are computed (e.g., glucose rate of change and/or Insulin On Board (IOB)). These obtained features are then fed to an unsupervised AD algorithm, which produces an anomaly score that measures how much the new data differ from the other previously observed ones. The specific criteria employed to assign the score can vary from one AD algorithm to the other. When the anomaly score exceeds a threshold, an alert is generated to cause generation of the prompt for command confirmation (e.g., process 106 in FIG. 6) and/or otherwise warn the patient of a possible malfunction of the FDD 10. If no user confirmation is received, then the command is ignored; otherwise, the FDD 10 is operated in accordance with the command.

The following is a non-limiting example of an anomaly detection algorithm employing a supervised machine-learning model to determine if a command is anomalous per process 102 in FIG. 6 in accordance with another example embodiment. This example supervised AD algorithm learns normal patient infusion patterns in terms of the dosage amount, rate, and time of infusion, which are automatically recorded in a FDD 10 logs or otherwise reported to a remote device for storage. In any event, these logs can be selected as a training data set to provide variables chosen from time, blood glucose (BG) levels, estimated bolus, target high BG, target low BG, carbohydrate (carb) ratio, carb input, food estimate, insulin sensitivity, active insulin or IOB, daily total insulin, basal rate, estimated bolus dose, correction estimate, among other variables and their associated time stamps. Further, as stated above, a log(s) can be transferred from one FDD 10 to the next FDD 10 worn by a patient. Also, an initial data set of the patient's relevant historical and/or current disease management data can be inputted from or via another device. It can be the responsibility of the FDD 10 user to start a bolus or change the basal rate manually. The patient or user can set the bolus dosages according to a control algorithm provided with the FDD 10, and set a basal rate as suggested by the patient's doctor.

This example supervised AD algorithm utilizes the temporal correlation of infusions for a particular patient. Patient-specific infusion patterns are captured over time, and the long term changes of infusion patterns can also be detected. A safety range can be dynamically updated at different times of the day. More specifically, generated regression models can be used to dynamically configure a safe infusion range for abnormal infusion command identification. For example, the regressions can be configured to analyze infusion dosage history and predict future infusion dosages and, once data is collected and analyzed after a selected period of time, a safety range for a specified time interval can be generated. Further, two sub models can be used for bolus (one type of insulin) abnormal dosage detection and basal abnormal rate detection.

This example supervised AD algorithm can detect anomalous commands for bolus dosage and/or basal rate, and also when total daily insulin is exceeded by a command. When a command is received per process 100 in FIG. 6, the AD algorithm checks whether the total daily insulin falls in the safety range. If the commanded insulin amount would fall out of the safety range, then the prompt per process 106 in FIG. 6 and/or an alert is generated for the patient. If the commanded insulin amount is within the safety range, then the sub model for abnormal bolus detection is used, or the sub model for abnormal basal rate detection can be used, depending on the commanded operation type. For example, a support vector machine (SVM) regression model can be used to predict bolus dosages using a vector representing, for example, time label, estimated bolus, target high BG, target low BG, carb ratio, insulin sensitivity, carb input, BG input, correction estimate, food estimate, active insulin and time, respectively, although a different set of more or fewer or different combination of variables can be used. SVM can also be used to predict basal rate and needs only a small record or patterns since basal patterns change less frequently. If either sub model detects an anomaly in the command, then a prompt is generated for a user to confirm the command. If no user confirmation is received, then the command is ignored; otherwise, the FDD 10 is operated in accordance with the command. This example supervised AD algorithm is convenient because it does not require any extra information since the required data is generally already present in an insulin pump log or via a related disease management data capture system or modality. Furthermore, the linear regression requires little memory and computing capacity, which can be done in real-time even on resource-limited computing platforms.

The AD algorithm(s) per process 102 in FIG. 6 and in accordance with example embodiments are beneficial because it can identify infusion mistakes made by doctors or patients, such as an erroneous dosage input, as well as identify an anomalous command from a malicious actor using a wireless interface to the FDD 10. For example, nefarious and potentially harmful commands to bolus dosages and basal rates could be sent to the FDD 10 by attackers (e.g., via a wireless link to the FDD 10). An attacker could issue a one-time overdose or underdose insulin amount to the patient, or could maintain a chronic attack wherein the attacker issues extra portions of medication to the patient over a long period of time. By performing anomaly detection, issuing a prompt for confirmation when an anomalous command is received, and ignoring the anomalous command unless a user confirmation of the command is received, the FDD 10 protects the patient from both user and doctor errors and malicious attacks that may not otherwise be discernible to the user (e.g., silent hacked anomalous commands). The example embodiments described herein are therefore advantageous over medical delivery devices without anomaly detection, including those that require users to confirm their command inputs, since these medical delivery devices cannot notify the user of inputs from another source like a malicous attacker. The example embodiments described herein are also advantageous over these medical delivery devices because it can be inconvenient to have to confirm every command input.

Typical drug delivery patch pump designs are challenged by the need to achieve small size, low power consumption, accurate delivery, high reliability, and low manufacturing costs. An example embodiment of the present disclosure achieves a technical solution to the technical problem of protecting a patient from dosing errors or other undesired medical device operations due to incorrect wireless commands; that is, from intentional nefarious interference with the wireless communication channel and command channel takeover, and/or from user operation error (e.g., intentionally or unintentionally entering an erroneous command for an anomalous operation of the medical device with respect to that patient). The example embodiments of the present disclosure can also achieve this technical solution while minimizing cost by providing means to enter confirmation of a command or to see an inputted command amount or rate of medication using a display on the auxiliary device 63 and without requiring the cost and complexity of an LED display or LCD on the FDD 10. In addition, example embodiments of the present disclosure further achieve this technical solution while minimizing cost by making the use of a different secondary confirmation channel optional, thereby omitting the need to use an NFC circuit at the FDD 10 for a secondary channel, for example.

Although various persons, including, but not limited to, a patient or a healthcare professional, can operate or use illustrative embodiments of the present disclosure, for brevity an operator or user will be referred to as a “user” hereinafter.

Although various fluids can be employed in illustrative embodiments of the present disclosure, for brevity the liquid in an injection device will be referred to as “fluid” hereinafter.

It will be understood by one skilled in the art that this disclosure is not limited in its application to the details of construction and the arrangement of components set forth in the above description or illustrated in the drawings. The embodiments herein are capable of other embodiments, and capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Further, terms such as up, down, bottom, and top are relative, and are employed to aid illustration, but are not limiting.

The components of the illustrative devices, systems and methods employed in accordance with the illustrated embodiments can be implemented, at least in part, in digital electronic circuitry, analog electronic circuitry, or in computer hardware, firmware, software, or in combinations of them. These components can be implemented, for example, as a computer program product such as a computer program, program code or computer instructions tangibly embodied in an information carrier, or in a machine-readable storage device, for execution by, or to control the operation of, data processing apparatus such as a programmable processor, a computer, or multiple computers.

A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network. Also, functional programs, codes, and code segments for accomplishing the illustrative embodiments can be easily construed as within the scope of claims exemplified by the illustrative embodiments by programmers skilled in the art to which the illustrative embodiments pertain. Method steps associated with the illustrative embodiments can be performed by one or more programmable processors executing a computer program, code or instructions to perform functions (e.g., by operating on input data and/or generating an output). Method steps can also be performed by, and apparatus of the illustrative embodiments can be implemented as, special purpose logic circuitry, e.g., an FPGA (field programmable gate array) or an ASIC (application-specific integrated circuit), for example.

The various illustrative logical blocks, modules, and circuits described in connection with the embodiments disclosed herein may be implemented or performed with a general purpose processor, a digital signal processor (DSP), an ASIC, a FPGA or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example, semiconductor memory devices, e.g., electrically programmable read-only memory or ROM (EPROM), electrically erasable programmable ROM (EEPROM), flash memory devices, and data storage disks (e.g., magnetic disks, internal hard disks, or removable disks, magneto-optical disks, and CD-ROM and DVD-ROM disks). The processor and the memory can be supplemented by, or incorporated in special purpose logic circuitry.

Those of skill in the art would understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Those of skill would further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of claims exemplified by the illustrative embodiments. A software module may reside in random access memory (RAM), flash memory, ROM, EPROM, EEPROM, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. In other words, the processor and the storage medium may reside in an integrated circuit or be implemented as discrete components.

Computer-readable non-transitory media includes all types of computer readable media, including magnetic storage media, optical storage media, flash media and solid state storage media. It should be understood that software can be installed in and sold with a central processing unit (CPU) device. Alternatively, the software can be obtained and loaded into the CPU device, including obtaining the software through physical medium or distribution system, including, for example, from a server owned by the software creator or from a server not owned but used by the software creator. The software can be stored on a server for distribution over the Internet, for example.

The above-presented description and figures are intended by way of example only and are not intended to limit the illustrative embodiments in any way except as set forth in the following claims. It is particularly noted that persons skilled in the art can readily combine the various technical aspects of the various elements of the various illustrative embodiments that have been described above in numerous other ways, all of which are considered to be within the scope of the claims.

1. A fluid delivery device comprising:
a reservoir that can contain a fluid to be delivered to a user;
a user output port configured to provide a fluid path between the reservoir and the user;
a drive assembly configured to controllably drive fluid from the reservoir into the fluid path;
a communication interface configured to send and receive signals on a communication path; and
a controller connected to the drive assembly and the communication interface and configured to control the drive assembly to deliver a designated amount of fluid from the reservoir to the fluid path;
wherein the controller is further configured to
analyze a command to operate the drive assembly to determine when the command corresponds to an anomalous command;
generate a prompt for confirmation of the command when the command corresponds to an anomalous command, operate the drive assembly in accordance with the command when a reply to the prompt is received and comprises an input corresponding to an affirmative confirmation, and ignore the command when the reply to the prompt is a negative confirmation chosen from an input corresponding to a negative confirmation, and no reply being received within a designated time period after the prompt.

2. The fluid delivery device of claim 1, wherein the controller is further configured to operate the drive assembly in accordance with the command without the prompt for confirmation or the reply with an input corresponding to an affirmative confirmation when the command is not an anomalous command.

3. The fluid delivery device of claim 1, wherein the controller is configured to process a parameter in the command in accordance with at least one anomaly detection algorithm to compare the parameter with a value chosen from a threshold value and a selected range of values, and to determine the command to be an anomalous command if the parameter exceeds the threshold value or is outside the selected range of values and to be normal command if the parameter satisfies a threshold value or selected range of threshold values.

4. The fluid delivery device of claim 3, wherein the parameter level or range is preset, or adapted over time in accordance with operation of the fluid delivery device.

5. The fluid delivery device of claim 1, wherein the controller is configured to process the command by analyzing variables chosen from historical commands to deliver the fluid to the patient and historical disease management data related to the patient to identify a fluid delivery device use pattern corresponding to one or more parameters related to amounts of fluid delivered, when the fluid is delivered, and patient physiological data affected by delivery of the fluid, and to detect when the command corresponds to an anomalous command because a parameter in the command is an outlier relative to one or more of the parameters of the use pattern.

6. The fluid delivery device of claim 1, wherein the controller determines when the command corresponds to an anomalous command by using at least one anomaly detection algorithm.

7. The fluid delivery device of claim 6, wherein the at least one anomaly detection algorithm employs anomaly detection techniques chosen from local outlier factor, histogram-based outlier score, one-class support vector machine, robust covariance, k-nearest neighbor, isolation forests, supervised machine-learning model, unsupervised machine-learning model, and hybrid approach using a semi-supervised machine-learning model.

8. The fluid delivery device of claim 1, wherein the command is communicated to the fluid delivery device from an auxiliary device.

9. The fluid delivery device of claim 8, wherein the auxiliary device is chosen from a smartphone, a computer, a laptop, an iPad, and a dedicated, portable remote controller configured to command the fluid delivery device.

10. The fluid delivery device of claim 8, wherein the auxiliary device is connected to the fluid delivery device by a communication modality chosen from WiFi, Bluetooth®, near field communication (NFC), cellular communication, and wired communication, and is configured to remotely control the fluid delivery device by sending commands via the communication modality.

11. The fluid delivery device of claim 8, wherein the auxiliary device is connected to the fluid delivery device by a communication modality, and the command and the prompt are both transmitted using the communication modality.

12. The fluid delivery device of claim 11, wherein the communication modality is chosen from WiFi, near field communication (NFC) and Bluetooth®.

13. The fluid delivery device of claim 8, wherein the prompt is transmitted using a second communication modality that is different from a first communication modality used to send the command.

14. The fluid delivery device of claim 13, wherein the first communication modality is chosen from WiFi and Bluetooth®, and the second communication modality is chosen from near field communication (NFC), a magnet and Hall Effect sensor provided to respective ones of the fluid delivery device and an auxiliary device that sends the command to the fluid delivery device, an accelerometer sensor configured to detect a movement of an auxiliary device relative to the fluid delivery device, and a switch on the fluid delivery device.

15. The fluid delivery device of claim 1, wherein the controller comprises a communication processor device and a safety processor device, wherein the communication processor device establishes secure messaging with an auxiliary device connected to the fluid delivery before passing a message signal comprising the command from the auxiliary device to the fluid delivery device, and the safety processor device performs anomaly detection of the command.

16. The fluid delivery device of claim 16, wherein the communication processor device and the safety processor device are both provided within the fluid delivery device.

17. The fluid delivery device of claim 1, further comprising a processing device separate from and coupled to the controller;
wherein the processing device is configured to analyze the command to operate the drive assembly to determine when the command corresponds to an anomalous command, and to provide an indication to the controller when the command is determined to correspond to an anomalous command; and
wherein the controller is configured to generate the prompt for confirmation of the command when the command corresponds to an anomalous command, operate the drive assembly in accordance with the command when a reply to the prompt is received and comprises an input corresponding to an affirmative confirmation, and ignore the command when the reply to the prompt is a negative confirmation chosen from an input corresponding to a negative confirmation, and no reply being received within a designated time period after the prompt.
